(19) United States
(12) Patent Application Publication
(10) Pub. No.: US 2014/0020452 A1
(43) Pub. Date: Jan. 23, 2014

(54) FUEL PROPERTY SENSOR

(71) Applicant: DENSO CORPORATION, Kariya-city (JP)

(72) Inventors: Masato UENO, Takahama-city (JP); Hiroshi NAKAMURA, Nishio-city (JP); Tatsuya KITANAKA, Nagoya-city (JP); Dai OSADA, Kariya-city (JP)

(21) Appl. No.: 13/916,014

(22) Filed: Jun. 12, 2013

(30) Foreign Application Priority Data
Jul. 19, 2012 (JP) 2012-160485

(51) Int. Cl. G01N 33/22

(52) U.S. Cl. CPC G01N 33/22
USPC 73/61.46

(57) ABSTRACT

A fuel property sensor has an external electrode, an internal electrode, a temperature detecting device, a holder portion, a sealing portion, and a calculation unit. The external electrode includes a passage portion and a projecting portion. The internal electrode is disposed in the passage portion of the external electrode at a predetermined distance from an inner wall of the passage portion and the internal electrode. The temperature detecting device detects a fuel temperature. The holder portion is disposed in the projecting portion of the external electrode and holds the temperature detecting device therein, and the holder portion and the projecting portion define a transfer passage. The sealing portion insulates the external electrode and the holder portion from each other, and prevents fuel flowing in the transfer passage from leaking outside. The calculation unit calculates fuel properties and electrical characteristics of fuel flowing between the external electrode and the internal electrode.

### CROSS REFERENCE TO RELATED APPLICATION

This application is based on Japanese Patent Application No. 2012-160485 filed on Jul. 19, 2012, the disclosure of which is incorporated herein by reference in its entirety.

### TECHNICAL FIELD

The present disclosure relates to a fuel property sensor that determines an alcohol concentration of fuel.

### BACKGROUND

Conventionally, a fuel property sensor detects an alcohol concentration of fuel. Specifically, the fuel property sensor calculates capacitance between two electrodes from a charge quantity of the two electrodes soaked in the fuel, and the alcohol concentration is calculated based on a relationship between the capacitance and a detected fuel temperature. For instance, JP-A-2009-505074 (U.S. Pat. No. 7,466,147) describes such a fuel property sensor in which a thermistor is disposed in an attachment portion that abuts a second electrode soaked in fuel, and a fluid quality sensor is used to detect a fuel temperature.

However, in the fluid quality sensor of JP-A-2009-505074, an insulator is located closer to the fuel than the thermistor is. The insulator insulates a first electrode from the attachment portion where the first electrode is located in a radial-outward direction of a second electrode and the attachment portion from each other. The insulator is exposed on the outside, so heat is transferred easily from external environment to the thermistor via the insulator.

### SUMMARY

According to an example of the present disclosure, there is provided a fuel property sensor having an external electrode, an internal electrode, a temperature detecting device, a holder portion, a sealing portion, and a calculation unit. The external electrode includes a passage portion, the passage portion defining a fuel passage through which fuel flows, and a projecting portion radially projecting from the passage portion. The internal electrode is disposed in the passage portion of the external electrode at a predetermined distance from an inner wall of the passage portion and the internal electrode. The temperature detecting device detects a fuel temperature. The holder portion is disposed in the projecting portion of the external electrode and holding the temperature detecting device therein. The holder potion is electrically coupled to the internal electrode, and the holder portion and the projecting portion define a transfer passage. The sealing portion is disposed between an outer wall of the holder portion and an inner wall of the projecting portion. The sealing portion insulates the external electrode and the holder portion from each other, and is disposed to prevent fuel flowing in the transfer passage from leaking outside. The calculation unit calculates fuel properties based on the fuel temperature detected by the temperature detecting device and calculates electrical characteristics of fuel flowing between the external electrode and the internal electrode. The passage portion and the projecting portion are connected such that fuel flowing in the fuel passage passes through the transfer passage. One end part of the sealing portion faces the passage portion. A center of the temperature detecting device is positioned between a central axis of the passage portion and the one end part of the sealing portion, in a section along a vertical axis that is perpendicular to the central axis in a radial direction of the passage portion.

### BRIEF DESCRIPTION OF THE DRAWINGS

The above and other objects, features and advantages of the present disclosure will become more apparent from the following detailed description made with reference to the accompanying drawings. In the drawings:

FIG. 1 is a schematic view of a fuel supply system that employs a fuel property sensor;

FIG. 2 is a cross-sectional view of the fuel property sensor;

FIG. 3 is a cross-sectional view taken along a line III-III of FIG. 2;

FIG. 4A is a cross-sectional view of the fuel property sensor; and

FIG. 4B is a graph showing a relationship between a location of the fuel seal disposed in the fuel property sensor and a detection error of a fuel temperature detected by a thermistor.

### DETAILED DESCRIPTION

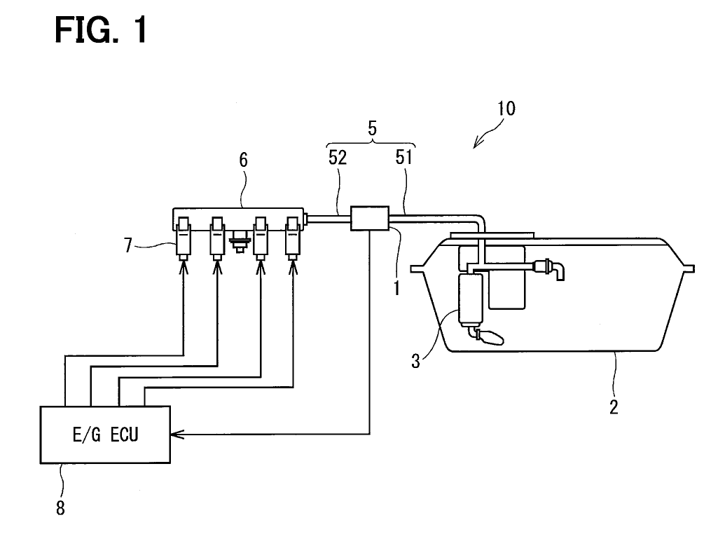

The present disclosure will now be described hereafter with reference to FIGS. 1-4B.

As shown in FIG. 1, a fuel supply system 10 that supplies the fuel to an internal combustion engine (not shown) has a fuel property sensor 1. The fuel property sensor 1 is disposed at a fuel pipe 5 that connects a fuel tank 2 and a delivery pipe 6. The fuel tank 2 stores fuel in which gasoline and ethanol are mixed. Gasoline, ethanol, or a mixture of gasoline and ethanol may be supplied to the fuel tank 2 as needed. Therefore, when gasoline, ethanol, or the mixture of gasoline and ethanol is supplied to the fuel tank 2, an ethanol concentration of fuel stored in the fuel tank 2 may change.

A fuel pump 3 pumps fuel stored in the fuel tank 2 to the delivery pipe 6 via the fuel pipe 5, and an injector 7 sprays the fuel into an intake pipe of a cylinder. An engine control unit (ECU) 8, which is a unit that controls an internal combustion engine, electrically controls actuation of the injector 7.

The ECU 8 includes a micro computer and the like. The fuel property sensor 1 transmits a signal to the ECU 8. Based on an ethanol concentration detected by the fuel property sensor 1, the ECU 8 controls various controlled parameters such as an air-fuel ratio, an amount of fuel consumption, and ignition timing appropriately. To actuate the internal combustion engine under optimum conditions, the fuel property sensor 1 may be disposed as close to the injector 7 as possible.

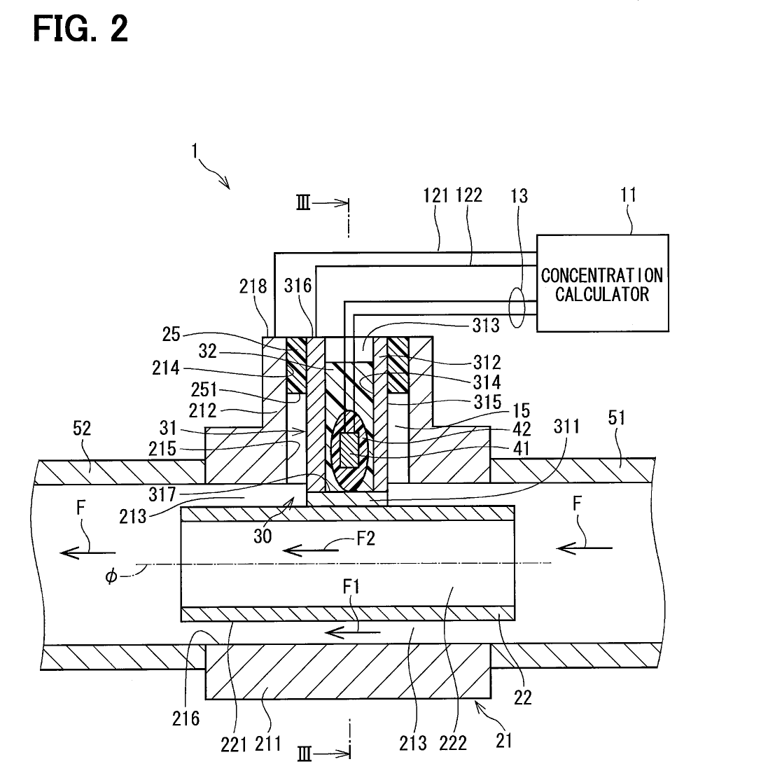

As shown in FIG. 2, the fuel property sensor 1 has an external electrode 21, an internal electrode 22, a temperature detecting part 30, and a concentration calculator 11 (i.e., a calculation unit). In FIG. 2, arrows F, F1 and F2 show the direction in which the fuel flows.

The external electrode 21 is a metallic member having a passage portion 211 and a cylinder portion 212 (i.e., a projecting portion), which are integrated to form the external electrode 21. The passage portion 211 has an opening at both of its ends, and the cylinder potion 212 is generally perpendicular to the passage portion 211.

The passage portion 211 is a cylindrical metallic member. The fuel pipe 5 includes a first pipe 51 and a second pipe 52. The passage portion 211 is disposed so that its central axis ø is in parallel with a fuel flow direction in the first pipe 51 and the second pipe 52 (i.e., parallel with F). A first opening at a first end of the passage portion 211 and the first pipe 51 are connected to transmit fuel. A second opening at a second end of the passage portion 211 and the second pipe 52 are connected to transmit fuel. The passage portion 211 defines a fuel passage 213. When fuel flows from the fuel tank 2 to the delivery pipe 6, a part of the fuel passes through the fuel passage 213 as shown by the arrow F1 in FIG. 2.

The cylinder portion 212 extends in a radial-outward direction of the passage portion 211. An end part 218 of the cylinder portion 212 is an end of the cylinder portion 212 that is farther from the central axis ø than the other end of the cylinder portion 212, and a conductive wire 121 is coupled to the end part 218. The conductive wire 121 is also coupled to the concentration calculator 11 electrically.

The external electrode 21 has a transfer passage 214 that penetrates the passage portion 211 to link the fuel passage 213 to the outside of the external electrode 21 via the transfer passage 214. The transfer passage 214 is disposed and extends to be perpendicular to the central axis ø. A temperature detecting part holder 31 (i.e., a holder portion), which holds the temperature detecting part 30, is disposed in the transfer passage 214.

The internal electrode 22 is a cylindrical metallic member that is located at the medial of the passage portion 211. The internal electrode 22 is positioned to have a predetermined distance between an outer wall 221 of the internal electrode 22 and an inner wall 216 of the passage portion 211. A central axis of the internal electrode 22 is coaxial with the central axis ø. The internal electrode 22 defines a fuel passage 222. When fuel flows from the fuel tank 2 to the delivery pipe 6, some of the fuel that flows outside of the fuel passage 213 passes through the fuel passage 222 as shown by the arrow F2 in FIG. 2. That is, in the fuel property sensor 1, fuel flows inside the first pipe 51 towards the second pipe 52 via the fuel passage 213 or the fuel passage 222.

The temperature detecting part 30 includes a thermistor 41 (i.e., temperature detecting device) and the temperature detecting part holder 31 holds the thermistor 41 by holding the temperature detecting part 30.

The thermistor 41 is a resistor body in which electrical resistance changes depending on ambient temperatures. The thermistor 41 is covered with a covering portion 42 made of resin and held in a holding space 313 that is defined by the temperature detecting part holder 31. A couple of conductive wires 13 connects the thermistor 41 and the concentration calculator 11.

The temperature detecting part holder 31 is a bottomed-cylindrical metallic member that has a bottom portion 311 and a cylinder portion 312, in which the bottom portion 311 is at a first end side of the cylinder portion 312. The temperature detecting part holder 31 is disposed in the transfer passage 214. The bottom portion 311 abuts with the outer wall 221 of the internal electrode 22, such that the temperature detecting part holder 31 and the internal electrode 22 are connected electrically. An inner bottom wall 317 of the bottom portion 311 (i.e., an inner bottom wall of the holder portion) abuts with the covering portion 42 that covers the thermistor 41.

The cylinder portion 312 is generally perpendicular to the central axis ø, and the cylinder portion 312 and the bottom portion 311 are integrated to define the holding space 313. The cylinder portion 312 has an opening on an opposite side of the bottom portion 311. A first end of a conductive wire 122, whose second end is connected to the concentration calculator 11, connects to an end part 316 of the cylinder portion 312 defining the opening. The couple of conductive wires 13 electrically connect the concentration calculator 11 and the thermistor 41 via the opening of the cylinder portion 312. A fuel seal 25 (i.e., a sealing portion) is disposed between the cylinder portion 312 and the cylinder portion 212 of the external electrode 21.

The fuel seal 25 is an annular resin body that is disposed between an outer wall 315 of the cylinder portion 312 and an inner wall 215 defining the transfer passage 214. The fuel seal 25 prevents fuel, which flows in the fuel property sensor 1, from leaking outside through the transfer passage 214, and isolates the external electrode 21 from the internal electrode 22.

An annular secondary fuel passage 15 is located on the central axis ø side of the fuel seal 25. For example, a first end part 251, which is an end part on the central axis ø side of the fuel seal 25, the outer wall 315 of the cylinder portion 312 and the inner wall 215 define the secondary fuel passage 15. That is, a part of the transfer passage 214 defines the secondary fuel passage 15. The secondary fuel passage 15 communicates with the fuel passage 213, and a part of fuel that flows in the fuel passage 213 is retained in the secondary fuel passage 15.

Gaps between the covering portion 42 of the thermistor 41 and the inner bottom wall 317 of the bottom portion 311, and between the covering portion 42 of the thermistor 41 and an inner wall 314 of the cylinder portion 312 (i.e., an inner wall of the holder portion) are filled with a thermally-conductive material 32. The thermally-conductive material 32 fixes the thermistor 41 in the temperature detecting part holder 31 and transfers heat from the bottom portion 311 and the cylinder portion 312 to the thermistor 41.

The concentration calculator 11 is a computer that has a central processing unit (CPU) (i.e., an operation unit), a read-only memory (ROM) (i.e., a memory unit) and a random access memory (RAM) (i.e., a memory unit). Based on charge quantities of the external electrode 21 and the internal electrode 22, a current signal is fed to the concentration calculator 11 via the conductive wires 121 and 122. A voltage signal is fed to the concentration calculator 11 via the couple of conductive wires 13 based on electrical resistance of the thermistor 41.

In the fuel property sensor 1, a capacitance of a capacitor changes based on electrical characteristics of the fuel that flows in the fuel passage 213 between the external electrode 21 and the internal electrode 22 (shown by the arrow F1 in FIG. 2). The capacitor is defined by the external electrode 21 and the internal electrode 22, through which the fuel passes. Based on the current signal provided to the concentration calculator 11 via the conductive wires 121 and 122, the concentration calculator 11 determines the capacitance. In addition, based on the voltage signal that is fed to the concentration calculator 11 via the couple of conductive wires 13, the concentration calculator 11 also determines a temperature of the area where the thermistor 41 is located. In the concentration calculator 11, based on the determined capacitance and the determined temperature, an ethanol concentration of the fuel is calculated. Information regarding the calculated ethanol concentration of the fuel is provided to the ECU 8.

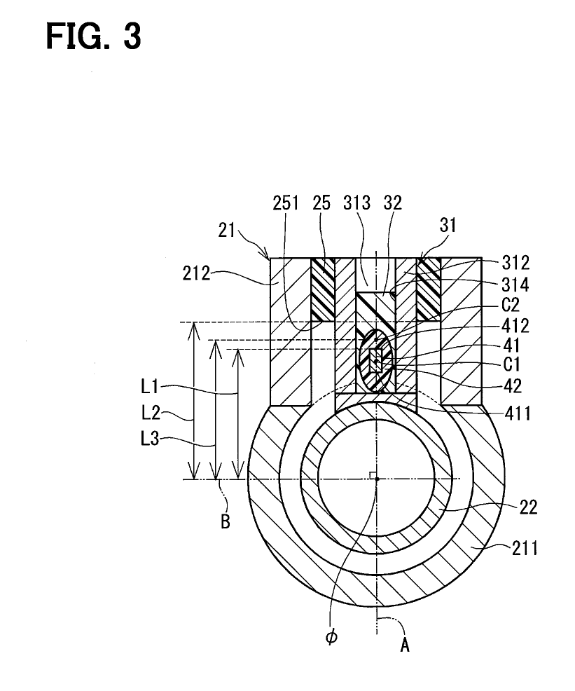

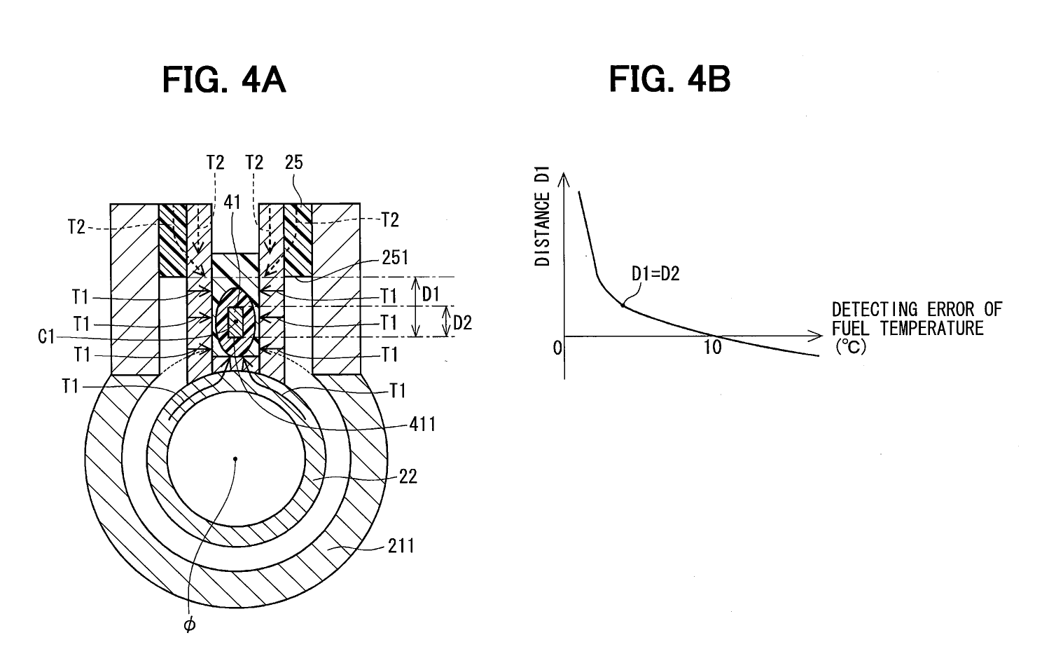

Positional relationships between the central axis ø and the thermistor 41 and between the thermally-conductive material 32 and the fuel seal 25 may be a part of main characteristics of the fuel property sensor 1. The positional relationships will be describes hereafter with reference to FIGS. 3-4B, and effects of the fuel property sensor 1 will be describes as well.

FIG. 3 is a cross-sectional view that is perpendicular to the fuel flow direction of the fuel property sensor 1.

In FIG. 3, suppose, for instance, there are a plane A and a plane B. The plane A passes through the central axis ø and a center C1 of the thermistor 41, and is in parallel to the fuel flow direction. The plane B passes through the central axis ø and is perpendicular to the plane A.

In the fuel property sensor 1, a distance L2 between the plane B and the first end part 251 of the fuel seal 25 is longer than a distance L1 between the central axis ø and a second end part 412 of the thermistor 41. The central axis ø is located on an opposite side of the first end part 251 with respect to the second end part 412 of the thermistor 41. In other words, the second end part 412 is in between the first end part 251 of the fuel seal 25 and the central axis ø.

Moreover, the distance L2 is longer than a distance L3 between the plane B and a center C2 of the thermally-conductive material 32. The central axis ø is located on an opposite side of the first end part 251 of the fuel seal 25 with respect to the center C2. In other words, the center C2 of the thermally-conductive material 32 is in between the central axis ø and the first end part 251 of the fuel seal 25.

FIG. 4B shows an examined result indicating a relationship between positions of the fuel seal 25 with respect to the thermistor 41 and a detection error of a fuel temperature detected by the thermistor 41.

Heat transfer to the thermistor 41 is shown schematically in FIG. 4A. T1 and T2 in FIG. 4A show such heat transferring directions. Solid arrows T1 show heat transfer from the fuel, and dashed arrows T2 show heat transfer from the external environment. FIG. 4B shows a relationship between a distance D1, between a third end part 411 of the thermistor 41 to the first end part 251 of the fuel seal 25, and the detection error of the fuel temperature. The third end part 411 of the thermistor 41 is the end part of the thermistor 41 that is close to the central axis ø. The graph of FIG. 4B shows the distance D1 on the y-axis and the detection error of the fuel temperature on the x-axis. In the graph, the third end part 411 of the thermistor 41 is the origin.

According to an experiment, when the distance D1 is 0, the detection error of the fuel temperature is 10° C. As shown in FIG. 4B, the longer the distance D1, the smaller the detection error of the fuel temperature. Especially, when the distance D1 is longer than a distance D2, between the third end part 411 and the second end part 412 of the thermistor 41, the detection error of the fuel temperature tends to be much smaller.

The fuel seal 25 of the fuel property sensor 1 is disposed between the cylinder portion 212 and 312, and a top surface of the fuel seal 25 is not covered so that heat may transfer easily from the external environment. The first end part 251 of the fuel seal 25 is disposed to be opposite to the central axis ø with respect to the second end part 412 of the thermistor 41. In other words, the fuel seal 25 is disposed, such that the second end part 412 is positioned between the fuel seal 25 and the central axis ø. Heat transferred from the external environment via the fuel seal 25 has an effect on the fuel temperature. By employing such a structure, the effect may be smaller than in a case where the first end part 251 of the fuel seal 25 and the central axis ø are on the same side with respect to the second end part 412 of the thermistor 41. Therefore, the detection error of the fuel temperature may decrease, thereby detecting the fuel temperature more accurately.

In the fuel property sensor, the first end part 251 of the fuel seal 25 is disposed to be opposite to the central axis ø with respect to the second end part 412 of the thermistor 41. By having such a structure, fuel flows in the secondary fuel passage 15, which is defined between the cylinder portion 212 of the external electrode 21 and the cylinder portion 312 holding the thermistor 41. Accordingly, the fuel between the cylinder portion 212 and 312 increases the capacitance. Thus, an absolute value of the capacitance detected by the fuel property sensor 1 is increased, and a signal to noise ratio (S/N) improves. Therefore, the detection accuracy of the fuel property sensor 1 improves.

The central axis ø is located on an opposite side of the first end part 251 of the fuel seal 25 with respect to the center C2 of the thermally-conductive material 32. That is, the center C2 of the thermally-conductive material 32 is located to face the secondary fuel passage 15, such that the position of the center C2 is adjacent to the secondary fuel passage 15. Moreover, a surface area of the thermally-conductive material 32 that is contacting the inner wall 314 of the cylinder portion 312 and in parallel to the secondary fuel passage 15 is larger than a surface area that is contacting the inner wall 314 of the cylinder portion 312 and in parallel to the fuel seal 25. By employing such a structure, most of the heat transferred to the thermistor 41 via the thermally-conductive material 32 may be from the fuel in the secondary fuel passage 15. Therefore, heat transferring from the fuel in the secondary fuel passage 15 to the thermistor 41 is dominant, and the detection error of the fuel temperature may decrease.

Although the central axis ø is on an opposite side of the first end part 251 of the fuel seal 25 with respect to the second end part 412 of the thermistor 41 in the embodiment, a positional relationship between the central axis ø and the first end part 251 is not limited to the above embodiment. The central axis ø may be located on an opposite side of the first end part 251 of the fuel seal 25 with respect to a center of the center C1 of the thermistor 41. By having such a structure, the center C1 of the thermistor 41 is positioned to face the secondary fuel passage 15. Therefore, when the thermistor 41 detects the fuel temperature, heat, which is transferred to the fuel via the fuel seal 25, has less effect on the determined fuel temperature than in a case where the center C1 of the thermistor 41 is positioned to face the fuel seal 25. Thus, the detection error of the fuel temperature may be reduced, and the fuel temperature is accurately detected.

Although the fuel temperature is detected by the thermistor 41 in the embodiment, a device that detects the fuel temperature is not limited to the above embodiment, and, for example, thermocouple may be applicable.

To sum up, the fuel property sensor of the present disclosure can be described as follows.

According to this disclosure, there is provided a fuel property sensor includes an external electrode, an internal electrode, a temperature detecting device, a holder portion, a sealing portion, and a calculation unit. The external electrode is constructed by a passage portion defining a fuel passage, in which fuel passes through, and a projecting portion projecting radially-outwardly from the passage portion. The internal electrode is disposed to have a predetermined distance in radial-inward direction of the passage portion between an inner wall of the passage portion and the internal electrode. The temperature detecting device detects a fuel temperature and feds into a signal depending on the detected fuel temperature. The holder portion that is inserted to a transfer passage and electrically connected to the internal electrode. The holder portion holds the temperature detecting device therein. The sealing portion is disposed between an outer wall of the holder portion and an inner wall defining the transfer passage. The holder portion insulates the external electrode and the holder portion from each other and prevents fuel flowing in the fuel passage from leaking outside. The calculation unit calculates fuel properties based on output signal, which is produced by the temperature detecting device, and electrical characteristics of fuel, which flows between the external electrode and the internal electrode. A central axis of the passage portion is located on an opposite side of one end part of the sealing portion, which is on the central axis side of the sealing portion, with respect to a center of the temperature detecting device. The external electrode defines the transfer passage that penetrates the passage portion and the projecting portion to communicate with both of the fuel passage and outside.

The holder portion may have heat, and the heat is transferred to the temperature detecting device. Especially, heat may be transferred to the holder portion from fuel around the holder portion and from the external environment via the sealing portion. The temperature detecting device detects the fuel temperature based on a sum of heat quantities transferred from the fuel and the external environment. Then, the central axis is located on an opposite side of the one end part of the sealing portion with respect to the center of the temperature detecting device. That is, the center of the temperature detecting device is located to be closer to the passage portion than the sealing portion is. Therefore, heat transferred from the external environment has less effect on the detected fuel temperature than that in case where the center of the temperature detecting device is located at further position from the passage portion than the sealing portion is. Thus, the detecting error of the fuel temperature is reduced, and the fuel temperature is detected more accurately.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A fuel property sensor comprising:
an external electrode including a passage portion, the passage portion defining a fuel passage through which fuel flows, and a projecting portion radially projecting from the passage portion;
an internal electrode disposed in the passage portion of the external electrode at a predetermined distance from an inner wall of the passage portion and the internal electrode;
a temperature detecting device detecting a fuel temperature;
a holder portion disposed in the projecting portion of the external electrode and holding the temperature detecting device therein, wherein the holder potion is electrically coupled to the internal electrode, and the holder portion and the projecting portion define a transfer passage;
a sealing portion disposed between an outer wall of the holder portion and an inner wall of the projecting portion, wherein the sealing portion insulates the external electrode and the holder portion from each other, and is disposed to prevent fuel flowing in the transfer passage from leaking outside; and
a calculation unit calculating fuel properties based on the fuel temperature detected by the temperature detecting device and calculates electrical characteristics of fuel flowing between the external electrode and the internal electrode; wherein
the passage portion and the projecting portion are connected such that fuel flowing in the fuel passage passes through the transfer passage,
one end part of the sealing portion faces the passage portion, and
a center of the temperature detecting device is positioned between a central axis of the passage portion and the one end part of the sealing portion, in a section along a vertical axis that is perpendicular to the central axis in a radial direction of the passage portion.

2. The fuel property sensor according to claim 1, wherein
one end part of the temperature detecting device is positioned between the central axis of the passage portion and the one end part of the sealing portion, in the section along the vertical axis.

3. The fuel property sensor according to claim 1, wherein
the holder portion has a thermally-conductive material disposed between the temperature detecting device and an inner surface of the holder portion, and
a center of the thermally-conductive material is positioned between the central axis of the passage portion and the one end part of the sealing portion.
